(19) United States
(12) Patent Application Publication
(10) Pub. No.: US 2021/0033528 A1
(43) Pub. Date: Feb. 4, 2021

(54) GAS ABSORPTION SPECTROSCOPIC DEVICE

(71) Applicant: Shimadzu Corporation, Kyoto (JP)

(72) Inventors: Hideaki KATSU, Kyoto (JP); Takashi MURAMATSU,, Kyoto (JP); Naoki MATSUDA, Kyoto (JP); Naoji MORIYA, Kyoto (JP)

(21) Appl. No.: 16/967,998

(22) PCT Filed: Jan. 15, 2019
(86) PCT No.: PCT/JP2019/000844

§ 371 Date: Aug. 6, 2020

(30) Foreign Application Priority Data
Feb. 19, 2018 (JP) 2018-026602

(51) Int. Cl. G01N 21/39; G01N 33/00; G01M 15/06

(52) U.S. Cl. CPC G01N 21/39; G01N 2201/0612; G01M 15/06; G01N 33/0036

(57) ABSTRACT

A gas absorption spectroscopic device is provided with a laser light source with a variable wavenumber 113, a photodetector 114 for detecting intensity of laser light emitted from the laser light source with a variable wavenumber and passed through a measurement target gas, and a laser driver 112 for supplying a driving current to the laser light source with a variable wavenumber 113 to repeatedly sweep the laser light at a predetermined wavenumber range. The gas absorption spectroscopic device is further provided with a pressure-related value acquisition means 117 for acquiring a pressure-related value which is a value of the pressure of the measurement target gas or a value varying in synchronism with the pressure, and a control means 131 for controlling the laser driver 112 to vary a wavenumber range for performing the sweep depending on the pressure-related value are provided. This enables high-precision measurement in a wide pressure range from low pressure to high pressure even in situations where the pressure of the measurement target gas varies and high-speed responsiveness is required.

### TECHNICAL FIELD

The present invention relates to a gas absorption spectroscopic device for measuring a concentration, a temperature, partial pressure, etc., of a specific gas contained in a measurement target gas based on a laser light absorption spectrum of the measurement target gas.

### BACKGROUND ART

As a gas absorption spectroscopy using a laser, the following two methods are mainly known.

(1) DLAS (Direct Laser Absorption Spectroscopy)

(2) WMS (Wavelength Modulated Spectroscopy)

In a DLAS, a measurement target gas is irradiated with laser light, and the laser light is measured by a photodetector. Here, there are two methods: a method of measuring absorption by gas with a wavelength (wavenumber) of laser light emitted to the gas fixed to a specified value; and a method of measuring an absorption spectrum of gas by sweeping a wavelength of laser light. In the former method, a wavelength of laser light is fixed to an absorption wavelength of a specific gas, and the absorbance at the wavelength is measured. When sweeping the wavelength, the spectrum is measured while changing the wavelength of the laser light within a range including the absorption wavelength of the specific gas, and the area of the gaseous absorption peak is measured.

The WMS resembles a wavelength-sweeping type DLAS, but in addition to the wavelength sweep, the WMS modulates the wavelength sinusoidally with a period sufficiently shorter than the sweep period (i.e., sufficiently higher frequency, which is denoted as “f” here). In the detector, it is possible to measure the absorption with higher sensitivity than the DLAS by detecting a higher harmonic of the frequency f (commonly used higher harmonic wave is a secondary higher harmonic). In the WMS, the gas concentration is easily calculated from the intensity of the acquired absorption spectrum.

In particular, as an industrial gas absorption spectroscopy, it is considered that a highly sensitive WMS is suitable. However, for the following reasons, the WMS has a problem that it is difficult to accurately perform gas measurement in high-speed measurement.

1. In order to perform the high-speed measurement, a wavelength modulation at high frequencies is required as well as shortening the sweep period. However, in the case of using an injection current control type wavelength-tunable diode laser which is generally most popular as a wavelength-tunable laser, when increasing the modulation frequency, the wavelength change rate with respect to the injection current decreases, so that a sufficient wavelength modulation width (modulation depth) cannot be acquired.

2. In particular, it is difficult to accurately measure a wavelength modulation width for high-speed modulations exceeding MHz, and an accurate wavelength modulation width cannot be determined for high-speed measurement. For this reason, the uncertainty of the information of the gas concentration, the temperature, etc., calculated from the measurement result increases.

In order to solve the above-described problems, some of the present inventors have proposed a new gas absorption spectroscopy in Patent Document 1 (hereinafter referred to as “improved WMS”). In this improved WMS, laser light is not modulated as in the above-described conventional WMS, but wavelength sweep of laser light is performed in a predetermined wavelength range including an absorption line of a specific gas in the same manner as in a wavelength sweep type DLAS. After passing through a measurement target gas, the light is received by a photodetector and its intensity change is detected. Since the wavelength range in which the wavelength sweep is performed is set to include the wavelength of an absorption line of a specific gas in advance, an absorption peak centered on the wavelength of the absorption line inherent in the specific gas appears in the spectral profile (curve of change in light intensity) of the light detected by the photodetector. In the improved WMS, the spectral profile containing this absorption peak is subjected to mathematical operations similar to the WMS processing. Specifically, centering on each wavelength point, an n^{th}-order polynomial approximation is performed for a spectral profile of a section corresponding to a wavelength modulation width of the WMS, and the WMS signal amplitude is reproduced using the coefficients of the n^{th}-order polynomial based on the Fourier transform principle. The principle is as follows.

Generally, in WMS processing, it is known that the spectral profile of the n^{th}-order harmonic acquired by the synchronous detection has a waveform acquired by approximately differentiating the absorption spectrum by the n^{th}-order (Non-Patent Document 1: Equation 8). Therefore, when the spectrum acquired by the wavelength sweep is differentiated by the n^{th}-order, it is considered that the spectrum corresponding to the n^{th}-order synchronous detection can be acquired. However, the effect of the noise of the measurement data becomes large when the n^{th}-order differential is performed, which is a problem in practical use. Therefore, in the improved WMS, the n^{th}-order polynomial approximation is performed for a certain range centering on the wavelength for which the harmonic signal is desired to acquire. The coefficients of the resulting polynomial become harmonic signals acquired by the WMS processing. In this case, the range in which the polynomial approximation is performed corresponds to the modulation amplitude in the WMS processing. The order of this approximate polynomial can be more accurately approximated with a higher order, but generally a first-order or second-order polynomial approximation is sufficient. In addition, the light quantity change correction processing such as light-blocking other than gas absorption is also performed.

In such an improved WMS, since only a wavelength sweep of several 100 kHz or less is performed in the light source, the oscillation wavelength for the injection current of the light source is precisely determined. Then, since the WMS processing is performed by mathematical operations based on the wavelength information, it is not affected by the nonlinearity of the light source driving power supply and the light source itself, which makes it possible to detect higher-order synchronization at an accurate wavelength modulation width.

### PRIOR ART DOCUMENT

### Patent Document

Patent Document 1: International Publication No. WO2014/106940

### Non-Patent Document

Non-Patent Document 1: Reid, J. and Labrie, D., “Second-harmonic detection with tunable diode lasers-comparison of experiment and theory,” Appl. Phys. B 26, 203-210 (1981)
Non-Patent Document 2: Katsuhiko Fukuzato, Yuji Ikeda, Ken Nakajima, “Measurement of CO₂ Gases Using Semiconductor-Laser Spectroscopy Systems (Second Report),” edited by the Japan Society of Mechanical Engineers, B edition, 2002, 68, 2901-2907
Non-Patent Document 3: G. B. Rieker, J. B. Jeffries, and R. K. Hanson, “Calibration-free wavelength modulation spectroscopy for measurements of gas temperature and concentration in harsh environments,” Appl. Opt., submitted 2009.

### SUMMARY OF THE INVENTION

### Problems to be Solved by the Invention

By the way, a center wavelength and a peak width of an absorption line acquired by a gas absorption spectroscopy depend on a pressure value of a measurement target gas. For example, the pressure property of the absorption line in the vicinity of an H₂O (concentration: 1.2%, optical path length: 1.0 cm, temperature: 400 K) of a wavelength 1,348.4 nm (wavenumber: 7,416.0 cm⁻¹) acquired from the HITRAN database is shown in FIG. 11. As is clear from the figure, as the gas pressure increases, the center position of the absorption line peak shifts, and the peak width increases.

Due to such pressure characteristics of the absorption line, a conventional gas absorption spectroscopy has a problem that the measurement accuracy in either the low pressure or the high pressure deteriorates in the situation where the pressure of the measurement target gas changes and the high-speed response is required. Specifically, for example, in order to reliably measure the absorption peak spread under high pressure, it is necessary to set the swept wavenumber (wavelength) range wide, in such a way, due to the limitation of the detection signal sampling rate, narrow peaks under low pressure cannot be measured with a high S/N ratio. On the other hand, when setting the sweep wavenumber range narrow so that a narrow peak can be measured with a high S/N ratio under low pressure, for the absorption peak spread under high pressure, only a portion can be measured, which results in a deteriorated S/N ratio, or the center of the peak deviates from the sweep wavenumber range due to the peak shift. As a result, there is a possibility that the desired physical quantity (e.g., specific gas concentration, etc.) cannot be measured.

The present invention has been made in view of the above-described points, and an object thereof is to provide a gas absorption spectroscopic device capable of performing highly accurate measurement in a wide pressure range from low pressure to high pressure in a situation where the pressure of a measurement target gas varies and high-speed responsiveness is required.

### Means for Solving the Problem

The gas absorption spectroscopic device according to the present invention made to solve the above-described problems includes:

a laser light source with a variable wavenumber;

a photodetector configured to detect intensity of laser light emitted from the laser light source with a variable wavenumber and passed through a measurement target gas;

a laser driving means configured to supply a driving current to the laser light source with a variable wavenumber to repeatedly sweep the laser light within a predetermined wavenumber range;

a pressure-related value acquisition means configured to acquire a pressure-related value, the pressure-related value being a pressure value of the measurement target gas or a value varying in synchronism with the pressure; and

a control means configured to control the laser driving means to vary the wavenumber range for the sweep depending on the pressure-related value.

Note that the “wavenumber” described here is intended to correspond uniquely to the “wavelength”, and it is, of course, also possible to assemble the same configuration using the “wavelength”.

According to the above configuration, based on the pressure-related value acquired by the pressure-related value acquisition means, by controlling the laser driving means so that the sweep wavenumber range becomes wider as the pressure of the measurement target gas increases, even in a situation where the pressure of the measurement target gas varies greatly from high pressure to low pressure, it is possible to perform wavenumber sweeping in an appropriate wavenumber range corresponding to the gas pressure at that time at all times. Therefore, the measurement can be performed with a high S/N ratio under both the low pressure and the high pressure.

Note that the gas absorption spectroscopic device and the method according to the present invention are applicable, for example, to the non-contact and high-speed measurement of a gas concentration, a temperature, and pressure in the automotive industry, and can be applied in a wide variety of fields such as gas measurement in high-temperature and high-pressure environments such as a combustion gas in a plant furnace. Here, for example, when the gas in a combustion chamber of an internal combustion engine or an external combustion engine of a piston-type is referred to as a measurement target gas, the value acquired by directly measuring the gas pressure in the combustion chamber with a pressure sensor can be used as the pressure-related value, and for example, a crank angle, which is a value that varies synchronously with the gas pressure in a combustion chamber, can be used as the pressure-related value.

The gas absorption spectroscopic device according to the present invention may further include a table storage means in which a plurality of tables defining different sweep waveforms different in sweep wavenumber width is stored in association with the pressure-related value, wherein the control means reads out a table corresponding to the pressure-related value acquired from the pressure-related value acquisition means among the plurality of tables from the table storage means and controls the laser driving means in accordance with the table.

The present invention can be applied, for example, to a gas absorption spectroscopic device configured to perform measurement by the above-described improved WMS. In the modified WMS, instead of modulating laser light, a light intensity change curve (spectral profile) detected by a photodetector is polynomially approximated by a wavenumber width corresponding to a modulation wavenumber width of the WMS at each point of the wavenumber. Note that it is known that the maximum S/N ratio is acquired when the “modulation wavenumber width of the WMS” is 2.2 times the half width at half maximum of the absorption line, but as described above, the peak width of the absorption line changes in accordance with the pressure of the measurement target gas. Therefore, in cases where the present invention is applied to a gas absorption spectroscopic device configured to perform measurement by the modified WMS, it is desirable to change not only the sweep wavenumber width but also the wavenumber width when the polynomial approximation is performed, according to the pressure change of the measurement target gas.

That is, the gas absorption spectroscopic device according to the present invention further includes

a polynomial approximation unit configured to approximate a curve of a change of light intensity detected by the photodetector by an approximate polynomial within a predetermined wavenumber width at each point of a wavenumber;

a differential curve generation unit configured to generate an n^{th}-order differential curve of the curve including the zero-order, based on a coefficient of each term of the approximate polynomial of each point; and

a physical quantity determination means configured to determine at least one of a temperature, a concentration, and partial pressure of a specific gas contained in the measurement target gas, based on the n^{th}-order differential curve including the zero-order,

wherein the polynomial approximation unit changes the wavenumber width to be approximated, depending on the pressure-related value acquired by the pressure-related value acquisition means.

Here, the “specific gas” denotes an optional ingredient determined by a measurer or the like, and is, for example, oxygen, water vapor, carbon dioxide, carbon monoxide, or the like.

Further, the present invention can be applied to a gas absorption spectroscopic device configured to perform measurement by the above-described WMS. In the WMS, the oscillation wavenumber of the laser is modulated at a predetermined frequency. At this time, the modulation wavenumber width at which the maximum S/N ratio is acquired varies according to the pressure of the measurement target gas. Therefore, in cases where the present invention is applied to a gas absorption spectroscopic device for performing measurement by the WMS, it is desirable to change not only the sweep wavenumber width but also the modulation wavenumber width, according to the pressure change of the measurement target gas.

That is, in the gas absorption spectroscopic device according to the present invention, the laser driving means modulates the driving current at a predetermined modulation amplitude and a predetermined modulation frequency. The gas absorption spectroscopic device further includes a demodulation means configured to extract a component of the modulation frequency or a harmonic component of the modulation frequency from a detection signal by the photodetector, and the control means further controls the laser driving means to vary the modulation amplitude depending on the pressure-related value.

### Effects of the Invention

As described above, according to the gas absorption spectroscopic device of the present invention, even in a situation where the pressure of a measurement target gas varies and high-speed responsiveness is required, the high-precision measurement can be performed over a wide pressure range from low pressure to high pressure.

### BRIEF DESCRIPTION OF THE DRAWINGS

FIG. 1 is a schematic configuration diagram of a gas absorption spectroscopic device according to an embodiment of the present invention.

FIG. 2 is a flowchart showing an operation of a control unit in the embodiment.

FIG. 3 is a schematic block diagram of a gas absorption spectroscopic device according to another embodiment of the present invention.

FIG. 4 is a flowchart showing an operation of an analysis unit in the embodiment.

FIG. 5 is a schematic block diagram of a gas absorption spectroscopic device according to still another embodiment of the present invention.

FIG. 6 is a flowchart showing an operation of a control unit in the embodiment.

FIG. 7 is a diagram showing a configuration example when gas in a combustion chamber of an engine is a measurement target gas.

FIG. 8 is an explanatory diagram schematically showing a method representing a spectral profile by polynomials in an improved WMS.

FIG. 9 is a waveform diagram showing a laser driving signal in the WMS, wherein (a) is a waveform of a sweeping signal, (b) is a waveform of a modulation signal.

FIG. 10 is a diagram showing a laser output waveform in the WMS.

FIG. 11 is a diagram illustrating a pressure-property of an absorption line of an H₂O having a wavelength of 1,348.4 nm (wavenumber: 7,416.0 cm⁻¹).

### EMBODIMENTS FOR CARRYING OUT THE INVENTION

### Embodiment 1

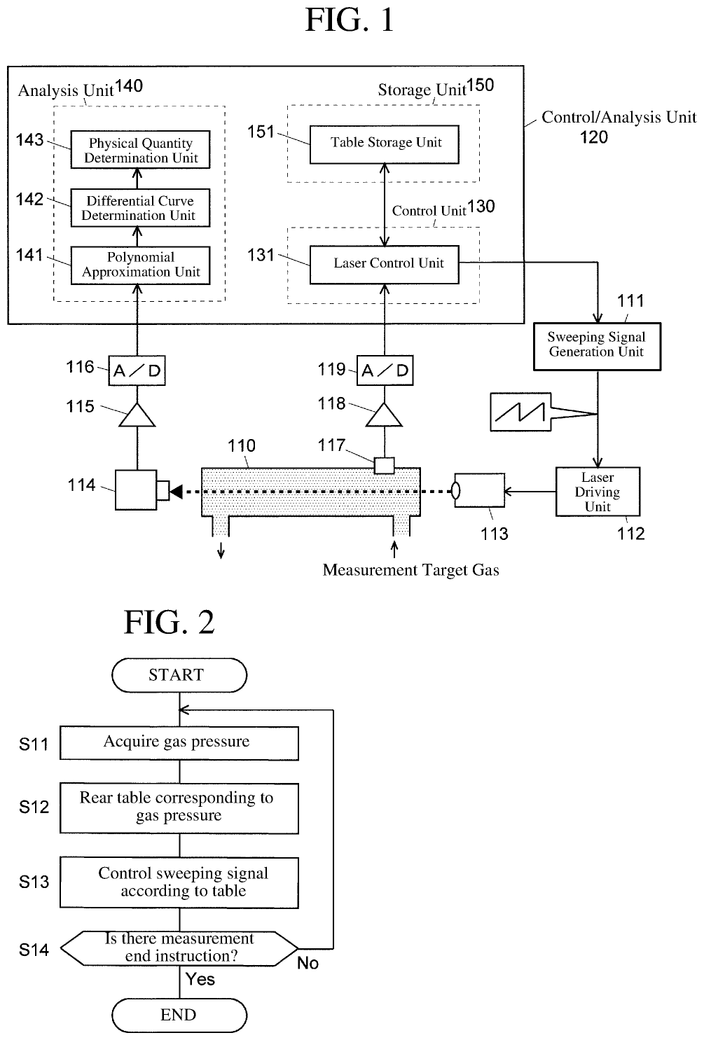

Hereinafter, an embodiment for carrying out the present invention will be described with reference to the drawings. The schematic configuration of a gas absorption spectroscopic device according to one embodiment of the present invention is shown in FIG. 1. The gas absorption spectroscopic device performs measurement by an improved WMS (i.e., the method described in Patent Document 1). The gas absorption spectroscopic device is provided with: a gas cell 110 through which a measurement target gas passes (or in which a measurement target gas is contained); a laser light source 113 and a photodetector 114 arranged to face with each other across the gas cell 110; a laser driving unit 112 for injecting a driving current into the laser light source 113; a sweeping signal generation unit 111 for inputting a predetermined sweeping signal to the laser driving unit 112; and a control/analysis unit 120 for controlling the above-described components and analyzing the outputs from the photodetector 114. Furthermore, the gas cell 110 is provided with a pressure sensor 117, which is one of the characteristic configurations in this embodiment.

The control/analysis unit 120 includes a control unit 130, an analysis unit 140, and a storage unit 150. The control unit 130 has a laser control unit 131 for controlling the sweeping signal generation unit 111. The analysis unit 140 includes a polynomial approximation unit 141 and a differential curve generation unit 142 for processing a detection signal of the photodetector 114, and a physical quantity determination unit 143 for calculating a desired physical quantity from the signal after the processing. The storage unit 150 is provided with a table storage unit 151 (which will be described in detail later).

The function of the control/analysis unit 120 is realized by a computer including a CPU, a memory, and a mass storage medium (such as a hard disk). The computer may be a dedicated computer built in the main body of the gas absorption spectroscopic device, but typically a personal computer or the like is used. A predetermined program is installed in the computer in advance, and the functions of the control unit 130 and the analysis unit 140 in this system are implemented by executing the program by the CPU. Further, the function of the storage unit 150 is realized by the mass storage medium.

The laser light source 113 is variable in wavenumber, and the wavenumber is swept in a predetermined wavenumber range including the wavenumber of the absorption line of the specific gas. Since the oscillation wavenumber of a semiconductor laser diode (hereinafter abbreviated as “LD”) used as a wavenumber tunable laser light source 113 depends on the magnitude of the injected current, the wavenumber sweeping of the laser light is performed by sweeping the injected current. Specifically, under the control of the laser control unit 131, the sweeping signal generation unit 111 generates a signal (see FIG. 1) having a sawtooth pattern, which is sent to the laser driving unit 112. The laser driving unit 112 injects an injection current serrated in accordance with the signal into the laser light source 113 composed of an LD. Thus, the oscillating wavenumber of the laser light source 113 is repeatedly swept in a predetermined wavenumber range.

First, the basic operation of the gas absorption spectroscopic device according to this embodiment will be described. When measuring the concentration, temperature, or partial pressure, etc., of a specific gas in a measurement target gas by the gas absorption spectroscopic device of this embodiment, under the control of the control unit 130, laser light of a predetermined maximum wavenumber is emitted from the laser light source 113, and the wavenumber is sequentially changed and swept to the lowest wavenumber. Note that in the above-described WMS, the wavenumber is modulated sinusoidally with a period sufficiently shorter than the sweep period in addition to the wavenumber sweeping, but the device according to this embodiment does not perform such modulation because the device performs measurement by the improved WMS. The light from the laser light source 113 passes through the measurement target gas in the gas cell 110 where the light is absorbed at the wavenumber of the absorption line of the specific gas. The laser light that has passed through the measurement target gas is detected its strength at the photodetector 114. The electric signal representing the light intensity, which is output from the photodetector 114, is digitally sampled by the A/D converter 116 via the amplifier 115 and sent to the analysis unit 140. The change in this electric signal becomes a spectral profile. The analysis unit 140 performs predetermined mathematical operations based on the data representing this spectral profile.

The mathematical operations performed by the analysis unit 140 will be described. Here, firstly, it is considered that the range [ν−a′<ν<ν+a′] of the width 2a′ of the above spectral profile, the range being centered on the respective points ν on the wavenumber axis, is represented by a polynomial represented by the following equation (1) (in the text of this application, an upper line attached to ν is represented by an underline in the text of this application due to the constraint of the electronic filing).

τ(ν)=b₀+b₁(ν−ν̅)+b₂(ν−ν̅)²+b₃(ν−ν̅)³+ . . . (1)

This is schematically shown in FIG. 8.

By acquiring the n^{th}-order derivative of the equation (1), the following can be acquired.

$$\frac{{d}^{n} τ ( v )}{d {v}^{n}} {\vert}_{v = \overline{v}} = {b}_{n} \qquad ( 2 )$$

Here, it is generally known that a spectral profile of an n^{th} harmonic acquired by synchronous detection by the WMS processing is approximately expressed by the following equation (Non-Patent Document 1: Equation 8).

$${H}_{n} ( \overline{v} ) \approx \frac{{2}^{1 - n} τ ( v )}{n !} {a}^{n} \frac{{d}^{n} τ ( v )}{d {v}^{n}} {\vert}_{v = \overline{v}} , n \geq 1 \qquad ( 3 )$$

Therefore, from the equations (2) and (3), the following can be acquired.

$${H}_{n} ( \overline{v} ) \approx \frac{{2}^{1 - n}}{n !} {a}^{n} {b}_{n} , n \geq 1 \qquad ( 4 )$$

Therefore, in order to calculate the WMS signal for the wavenumber ν in the above spectral profile, the range of the wavenumber of [ν̅−a′<ν+a′] is fitted by the least-squares method, etc., and the coefficients b₀, b₁, b₂, b₃ . . . are acquired. The profile of the coefficients b₁ and b₂ acquired by fitting with sequential variations of ν̅ corresponds to the WMS profile of 1f and 2f. Note that “a′” representing the range of the fitting is a value corresponding to a modulation amplitude “a” (i.e. modulation wavenumber width) of the WMS.

Specifically, the polynomial approximation unit 141 fits the range [ν−a′<ν<ν+a′] of the width 2a′ centering on the wavenumber ν of the spectral profile by the least-squares method or the like to acquire the coefficients b₀, b₁, b₂, b₃ . . . .

Subsequently, the differential curve generation unit 142 generates a profile of each coefficient (i.e., a higher-order differential curve including zero-order) by plotting the coefficients b₀, b₁, b₂, b₃ . . . acquired by performing the fitting by sequentially changing the above-mentioned wavenumber ν with respect to the wavenumber ν. Here, the profile of the coefficient b₁ and the profile of the factor b₂ correspond to the primary synchronous detection profile and the secondary synchronous detection profile in the WMS, respectively.

Subsequently, the physical quantity determination unit 143 calculates a concentration, partial pressure, a temperature, and the like of a specific gas in a measurement target gas based on the high-order differential curve (including zero-order) generated by the above-described processing. For example, a concentration of a specific gas can be calculated from the area of the absorption peak of a zero-order differential curve. The concentration of the specific gas can also be calculated from the peak height of the second-order differential curve. It is known that the partial pressure P of the measurement target gas has the following relation with the half-width α_{L} of the absorption peak of the zero-order differential curve.

$${α}_{L} = {α}_{L 0} ( \frac{P}{{P}_{0}} ) {( \frac{{T}_{0}}{T} )}^{γ} \qquad ( 5 )$$

Where α_{L0} is the Lorentz spread half width half maximum at the pressure P₀ and the temperature T₀, P₀ is the pressure of the measurement target gas at the reference time, T is the temperature of the measurement target gas at the time of measurement, T₀ is the temperature of the measurement target gas at the reference time, and γ is the constant representing the temperature dependency of the Lorentz width. The partial pressure of the specific gas can be determined by this equation. Further, as for the temperature of the specific gas, it is known that the ratio of the magnitudes of the two absorption peaks changes as the temperature changes, and by using the relationship, the temperature of the measurement target gas can be detected (Non-Patent Document 2).

Next, the normalization processing of the transmission light intensity will be described. One of the practical issues in the gas absorption spectroscopy is that the light intensity changes with contamination of the optical components used in the gas cell and with changes in the optical axis caused by vibrations in poor conditions. Therefore, although correction processing of the light intensity is required, as one of correction methods, normalization processing (Non-Patent Document 3) in which a synchronously detected 2f signal is divided by a 1f signal is known. In this method, however, in addition to the need to modulate the laser light, two types of synchronous detection circuitries must also be provided for 1f and 2f.

On the other hand, in the WMS corresponding processing using the polynomial approximation as described above, the modulation circuit and/or the synchronous detection circuit of the laser light is also not required, since the detection signals of 1f and 2f are calculated at the same time when performing the approximation, normalization processing can be performed very conveniently. Details will be given below.

When the light intensity entering into the measurement target gas is an I₀, the detected light intensity is S(ν)=GI₀ τ(ν). G denotes the drop (and variation) of the light intensity by each optical component and the electric gain for the detected light intensity. Therefore, in an actual apparatus, the WMS processing by the mathematical operation is applied to S(ν), and the following expression is acquired.

S(ν)=b₀′+b₁′(ν−ν̅)+b₂′(ν−ν̅)²+b₃′(ν−ν̅)³+ . . . (6)

Therefore, the coefficients acquired here will be as follows.

b₀′=GI₀b₀ (7a)

b₁′=GI₀b₁ (7b)

b₂′=GI₀b₂ (7c)

Therefore, in order to acquire a value that depends only on the transmission spectrum without depending on the variation of the light intensity, b₂′ (2f signal) may be divided by b₁′ (1f signal) or b₀′ as follows.

$$\begin{matrix}\frac{{b}_{2}^{′}}{{b}_{1}^{′}} = \frac{{b}_{2}}{{b}_{1}} & ( 8 a ) \\ \frac{{b}_{2}^{′}}{{b}_{0}^{′}} = \frac{{b}_{2}}{{b}_{0}} \approx {b}_{2} & ( 8 b )\end{matrix}$$

Note that since b_{0˜}1 (b₀ is close to 1) when the absorptivity is small, it can be approximated as shown by the expression (8b). From the above, it becomes possible to enable robust gas measurement independent of the light intensity.

Next, the characteristic operation in the gas absorption spectroscopic device of this embodiment will be described. As described above, the absorption line peak-width of the specific gas varies depending on the measurement target gas pressure. Therefore, in the gas absorption spectroscopic device according to this embodiment, the laser light source 113 is controlled so that the sweep wavenumber range is relatively wide when the pressure of the measurement target gas detected by pressure sensor 117 is high and the sweep wavenumber range is relatively narrow when the pressure is low so that the wide absorption peak under high pressure and the narrow absorption peak under low pressure can also be appropriately detected in the situation where a high-speed response is required.

In order to realize the above-described control, the table storage unit 151 stores a plurality of tables in which the sweep waveforms in which the sweep wavenumber widths differ for each measurement target gas pressure range are specified. The plurality of tables (hereinafter referred to as “table set”) is prepared for each type of a specific gas to be measured by the gas absorption spectroscopic device, and for example, when the user sets the type of the specific gas prior to starting the measurement, the table set corresponding to the type of the specific gas is automatically selected.

During the measurement of the measurement target gas, the electric signal representing the pressure of the measurement target gas, which is output from the pressure sensor 117, is digitally sampled by the A/D converter 119 via the amplifier 118 and sent to the control unit 130. Hereinafter, the operation of the control unit 130 will be described with reference to the flowchart of FIG. 2.

When the control unit 130 receives a signal representing the pressure (gas pressure) of the measurement target gas (Step S11), a table corresponding to the pressure is read out from the table storage unit 151 by the laser control unit 131 provided in the control unit 130 (Step S12). The laser control unit 131 controls the sweeping signal generation unit 111 based on the information of the sweep waveform described in the table (e.g., the value of the injected current at the respective times of the wavenumber sweeping) (Step S13). With this, the wavenumber sweeping of the laser light by the appropriate sweep wavenumber width according to the gas pressure detected by the pressure sensor 117 is performed. Such processing (Step S11 to S13) is repeatedly performed at regular intervals until an instruction to end the measurement is inputted from the user to the control/analysis unit 120 (i.e., until it becomes YES in Step S14).

Through the above-described processing, according to the gas absorption spectroscopic device of this embodiment, even in situations where the pressure of the measurement target gas varies and the high-speed responsiveness is required, wavenumber sweeping of the laser light at an appropriate sweep wavenumber width corresponding to the gas pressure at that time is performed, so that measurement can be performed at a constantly high S/N ratio.

Simulations performed to confirm the effects of the present invention will be described. Here, it is assumed that the voltage signal from the photodetector 114 input to the A/D converter 116 is a sawtooth signal having an H₂O absorbing profile of 1,348 nm, the sawtooth voltage value is 1 V to 4 V, and the noise width is 500 uVrms. Specifically, 32 types of signals formed by adding different white noise were prepared to the absorption line of 1,348 nm based on HITRAN2012, respectively, when the A/D converter 116 is assumed to acquire a digital value of 400 points in one sweep, when the average of “second-order coefficient/zero-order coefficient” calculated from the 32 types of data is S(=Signal) and the variance is N(=Noise), the S/N ratio was calculated. Consequently, at the low pressure (1 atm), when the modulation-width is 0.15 cm⁻¹ and the sweep-width is 2 cm⁻¹, the wavenumber sweeping is performed, the S/N ratio was 20 (S/N=20). On the other hand, at the low pressure (1 atm), when the modulation-width is similarly 0.15 cm⁻¹ and the sweep-width is 0.7 cm⁻¹, the S/N ratio was 33 (S/N=33). From this, it was confirmed that a higher S/N ratio was achieved by relatively narrowing the sweep width under low pressure.

### Embodiment 2

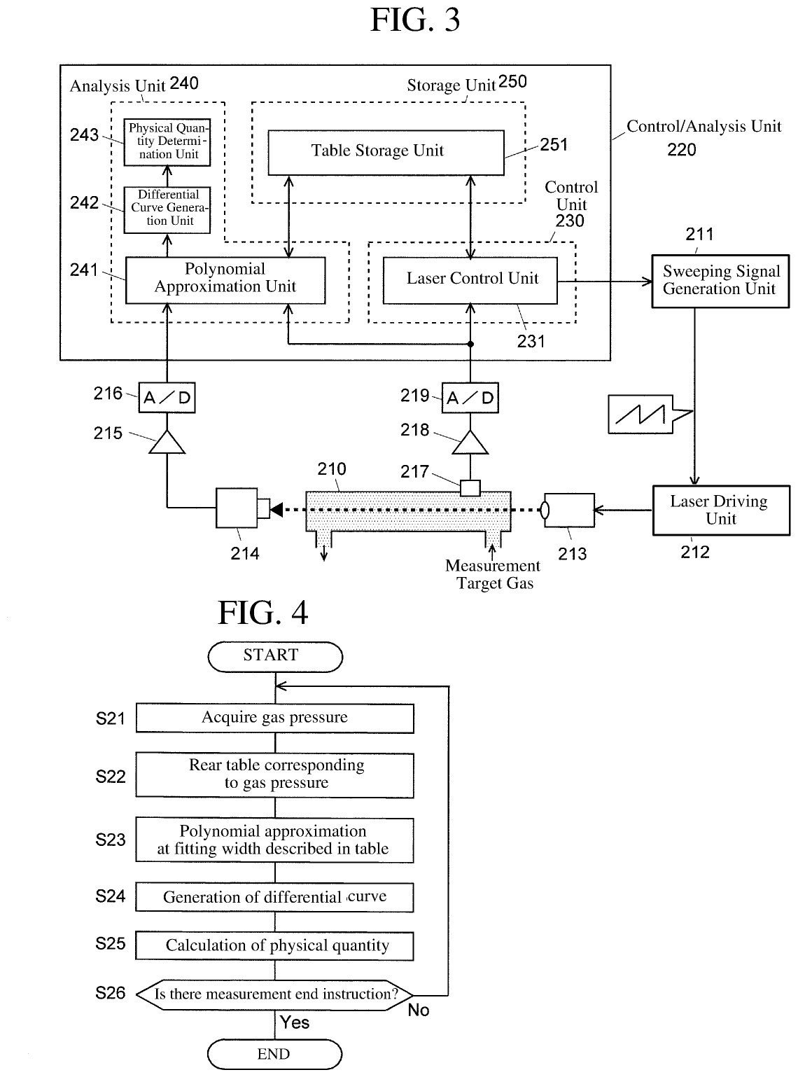

Hereinafter, another embodiment of the gas absorption spectroscopic device according to the present invention will be described with reference to FIG. 3 and FIG. 4. FIG. 3 is a diagram showing the schematic configuration of the gas absorption spectroscopic device according to this embodiment. FIG. 4 is a flowchart showing the operation of the analysis unit in the gas absorption spectroscopic device according to this embodiment. In this embodiment, the same or corresponding constituent elements as those described in FIG. 1 are denoted by the same reference numerals for the last two digits, and descriptions thereof are omitted as appropriate.

The gas absorption spectroscopic device according to this embodiment is intended not only to vary the sweep wavenumber width of the laser light but also to vary the fitting width when polynomially approximating the spectral profile at the analysis unit 240, in accordance with the pressure change of the measurement target gas. Therefore, in the gas absorption spectroscopic device according to this embodiment, the detection signal from the pressure sensor 217 is input not only to the control unit 230 but also to the analysis unit 240. Further, in the table storage unit 251, a plurality of tables (hereinafter, referred to as “table set”) is stored for each pressure range of the measurement target gas, similarly to Embodiment 1. In each table, in addition to the information of the sweep waveform suitable for measurement in the pressure range, the information of fitting widths suitable for polynomial approximations of the spectral profile acquired by measurement in the pressure range. Such a table set is prepared for each type of a specific gas to be measured by the gas absorption spectroscopic device, and, for example, when the user sets the type of a specific gas prior to starting the measurement, a table set corresponding to the type of the specific gas is automatically selected.

In this embodiment, the electric signal representing the pressure of the measurement target gas, which is output from the pressure sensor 217 during the measurement of the measurement target gas, is digitally sampled by the A/D converter 219 via the amplifier 218 and sent to the control unit 230 and the analysis unit 240. Hereinafter, the operation of the analysis unit 240 at this time will be described with reference to the flowchart of FIG. 4 (since the operation of the control unit 230 is the same as that shown in the flowchart of FIG. 2, descriptions thereof will be omitted).

The signal representing the pressure (gaseous pressure) of the measurement target gas is sent to the polynomial approximation unit 241 provided in the analysis unit 240 (Step S21). The polynomial approximation unit 241, which received the signal, reads out the table corresponding to the gas pressure from the table storage unit 251 (Step S22) and performs a polynomial approximation of the spectral profile with the fitting widths described in the table (Step S23). Based on the result, a differential curve is generated by the differential curve generation unit 242 (Step S24), and a specific gas concentration, a temperature, partial pressure, and the like is calculated by the physical quantity determination unit 243 (Step S25). Such processing (Steps S21 to S25) are repeatedly performed at regular intervals until an instruction to end the measuring is input from the user (i.e., until it becomes YES in Step S26).

As described above, in the improved WMS, instead of modulating the laser light, the curve of the change in light intensity (spectral profile) detected by the photodetector is polynomially approximated within the wavenumber width corresponding to the modulation wavenumber width of the WMS at each point of the wavenumber. At this time, the modulation wavenumber width of the WMS where the maximum S/N ratio is acquired varies according to the pressure of the measurement target gas. Therefore, in accordance with the gas pressure acquired by the pressure sensor 217 as described above, by changing the wavenumber width (fitting width) to perform the polynomial approximation from time to time, even in situations where the pressure of the measurement target gas varies and high-speed responsiveness is required, it is possible to constantly achieve a high S/N ratio.

### Embodiment 3

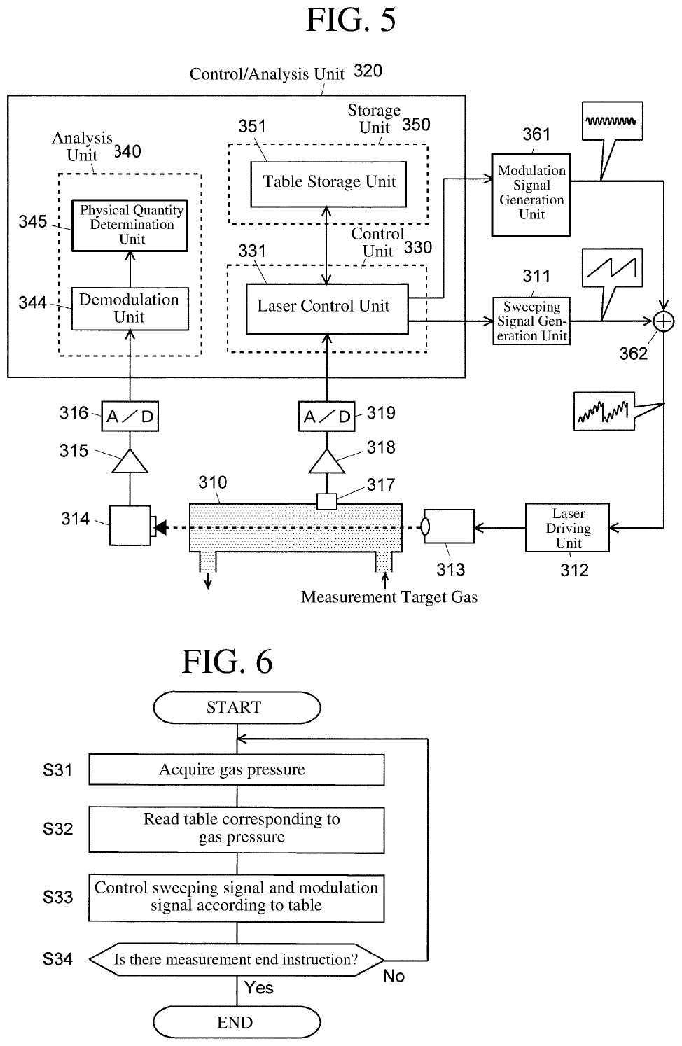

Hereinafter, a still another embodiment of the gas absorption spectroscopic device according to the present invention will be described with reference to FIG. 5 and FIG. 6. FIG. 5 is a diagram showing the schematic configuration of the gas absorption spectroscopic device according to this embodiment. FIG. 6 is a flowchart showing the operation of the control unit in the gas absorption spectroscopic device according to this embodiment. In this embodiment, the same or corresponding constituent elements as those described in FIG. 1 are denoted by the same reference numerals for the last two digits, and descriptions thereof are omitted as appropriate.

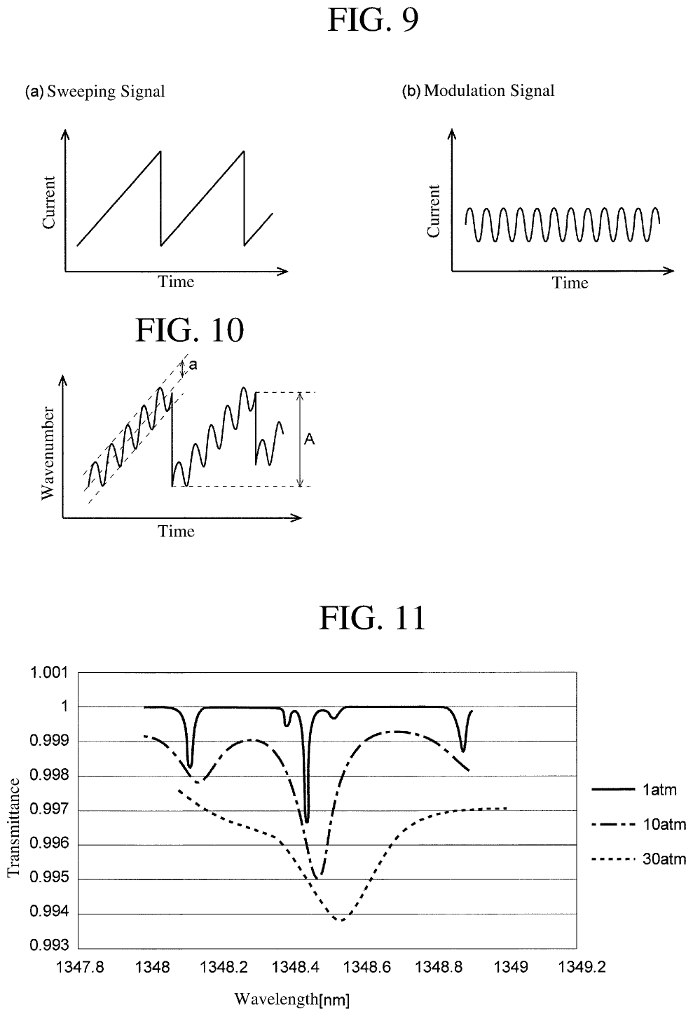

The gas absorption spectroscopic device is for measuring by the WMS, and is provided with, in addition to the sweeping signal generation unit 311 which generates a sawtooth sweeping signal similar to Embodiments 1 and 2, a modulation signal generation unit 361 for generating a modulation signal which is a sine wave having a higher frequency than the sawtooth sweeping signal. The sweeping signal (FIG. 9 (a)) and the modulated signal (FIG. 9 (b)) generated by the sweeping signal generation unit 311 and the modulation signal generation unit 361 respectively under the control of the control unit 330 are added in the adder 362 and sent to the laser driving unit 312. The laser driving unit 312 injects a sawtooth injected current modulated in accordance with the signal into the laser light source 313. With this, the output from the laser light source 313 is swept repeatedly at a predetermined sweep wavenumber width A and modulated at a predetermined modulation wavenumber width a, as shown in FIG. 10.

The control/analysis unit 320 has a control unit 330, an analysis unit 340, and a storage unit 350. The control unit 330 is provided with a laser control unit 331, and the analysis unit 340 is provided with a demodulation unit 344 for demodulating a detection signal of the photodetector 314 and a physical quantity determination unit 345 for calculating a desired physical quantity from the signal after the demodulation. The storage unit 350 is provided with a table storage unit 351 that stores a plurality of tables (table set) for each pressure range of the measurement target gas. In each table, the information of the sweep waveform and a modulation waveform that are suitable for measuring in that pressure range are written. The table set as described above is prepared for each type of the specific gas to be measured by the gas absorption spectroscopic device, and, for example, when the user sets the type of the specific gas prior to starting the measurement, the table set corresponding to the type of the specific gas is automatically selected.

These control unit 330 and analysis unit 340 are functional means realized as software by installing and executing dedicated software on a computer including a CPU, a memory, a mass storage device, and the like. The function of the storage unit 350 is realized by the mass storage medium.

In this embodiment, the electric signal representing the pressure of the measurement target gas, which is output from the pressure sensor 317 during the measurement of the measurement target gas, is digitally sampled by the A/D converter 319 via the amplifier 318 and sent to the control unit 330. Hereinafter, the operation of the control unit 330 will be described with reference to the flowchart of FIG. 6.

When the control unit 330 receives a signal representing the pressure (gas pressure) of the measurement target gas (Step S31), the table corresponding to the pressure is read out from the table storage unit 351 by the laser control unit 331 of the control unit 330 (Step S32). The laser control unit 331 controls the sweeping signal generation unit 311 and the modulation signal generation unit 361 according to the information of the seep waveform and the modulation waveform described in the table (Step S33). With this, wavenumber sweeping of the laser light by an appropriate sweep wavenumber width according to the gas pressure detected by the pressure sensor 317 and modulating of the laser light by an appropriate modulation wavenumber width according to the gas pressure is performed. Such processing (Steps S31 to S33) is repeatedly performed at regular intervals until an instruction to end the measuring is input from the user (i.e., until it becomes YES in Step S34).

As described above, in the gas absorption spectroscopic device according to this embodiment, by changing the sweep wavenumber width and the modulation wavenumber width of the laser light at any time according to the gas pressure acquired by the pressure sensor 317, even in a situation where the pressure of the measurement target gas varies and high-speed responsiveness is required, it is possible to constantly achieve a high S/N ratio.

Note that in this embodiment, the sweep wavenumber width and the modulation wavenumber width are changed in accordance with the gas pressure. However, only one of the sweep wavenumber width and the modulation wavenumber width may be changed in accordance with the gas pressure.

While this embodiment for carrying out the present invention has been described with specific examples, the present invention is not limited to the above-described embodiment, and modifications can be appropriately made within the spirit of the present invention.

For example, in Embodiments 1 to 3, the gas passing through the gas cell (or gas contained in the gas cell) is a measurement target gas, but instead of the above, the gas in the combustion chamber of the internal combustion engine or the external combustion engine may be a measurement target gas. In this case, it may be configured such that the pressure sensor is arranged in the combustion chamber, and the sweep wavenumber width of the laser light irradiated to the measurement target gas, the modulation amplitude of the laser light, or the fitting width of the spectral profile (hereinafter collectively referred to as “sweep wavenumber width or the like”) is changed in accordance with the value of the gas pressure acquired by the pressure sensor. However, when the internal combustion engine or the external combustion engine is of a piston type, it may be configured such that a pressure sensor is not be provided and the sweep wavenumber width or the like is changed in accordance with the rotational angle (i.e., crank angle) of the crankshaft rotating in accordance with the vertical movement of the piston. In this situation, the crank angle corresponds to the pressure-related value in the present invention.

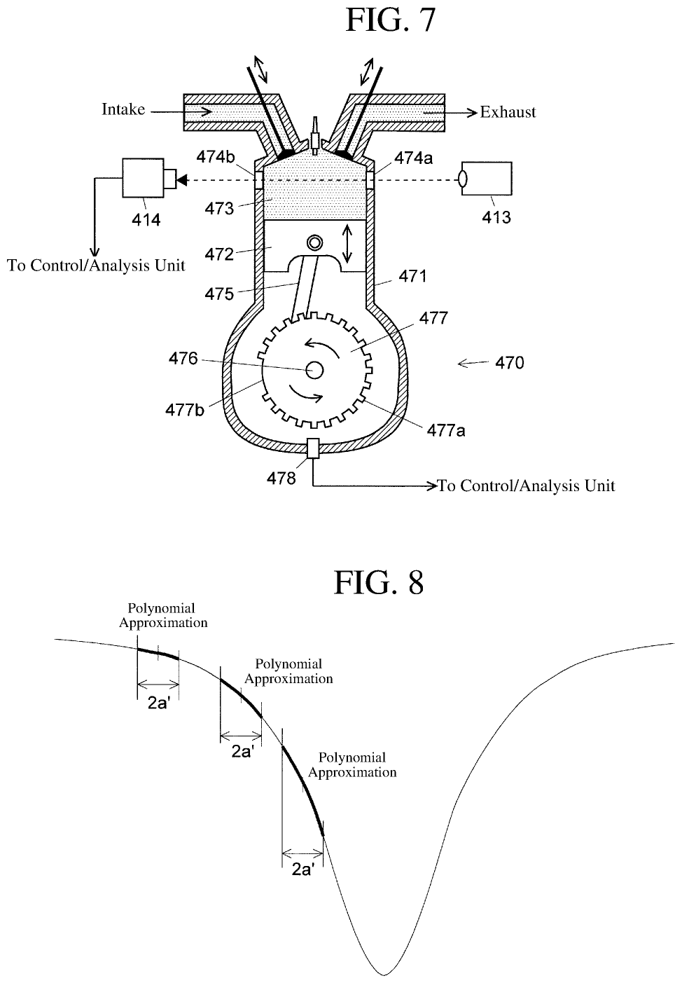

FIG. 7 shows a configuration example when the gas absorption spectroscopic device related to the present invention is applied to an engine of a piston type. The engine 470 has a cylinder 471 and a piston 472 slidable within the cylinder 471. A combustion chamber 473 is formed by the interior space of the cylinder 471 and the piston 472. Two optical windows 474a and 474b composed of lenses or the like are disposed opposite to each other on the peripheral surface of the cylinder 471, and the laser light emitted from the laser light source 413 enters the combustion chamber 473 from one of the optical windows 474a and then exits from the other optical window 474b to be received by the photodetector 414. The piston 472 is connected to a crankshaft 476 via a connecting rod 475. A timing rotor 477 having a plurality of protrusions 477a on the outer periphery is inserted in the crankshaft 476. The timing rotor 477 is configured to rotate with the rotation of the crankshaft 476. In the vicinity of the outer periphery of the timing rotor 477, a crank angle sensor 478 is arranged. The crank angle sensor 478 detects the protrusion 477a of the timing rotor 477 electromagnetically or optically and outputs a pulsed signal.

The crank signal (and a detection signal from the photodetector 414) from the crank angle sensor 478 is sent to a control/analysis unit which is not shown. Since there is an area 477b (missing tooth portion) in which the protrusion 477a is not provided in a part of the outer periphery of the timing rotor 477, a period in which no pulse appears also in the crank time periodically appears in the crank signal. Therefore, the control/analysis unit can identify the present crank angle by counting the pulses on the crank signal based on this time period. Since this crank angle varies synchronously with the gas pressure in the combustion chamber 473, the gas pressure in the combustion chamber 473 can be estimated based on the crank angle.

As the control/analysis unit, for example, a configuration similar to any one of control/analysis units 120, 220, and 320 in Embodiments 1 to 3 can be adopted. However, in any of the cases, a signal from the crank angle sensor 478 (crank signal) is used instead of the detection signal from the pressure sensor 117, 217, or 317, and the sweep wavenumber width or the like is changed based on the crank signal. In this case, the gas pressure in the combustion chamber 473 may be estimated from the count number of the crank signal as described above, and the sweep wavenumber width or the like may be changed in accordance with the gas pressure, but a plurality of tables describing an appropriate sweep wavenumber width or the like in each count number may be stored in the table storage unit 151, 251, or 351 in advance. In this case, a table corresponding to the count number acquired from the crank signal at each point during the measurement is read out from the table storage unit 151, 251, or 351 by the laser control unit 131, 231, or 331, and the sweep wavenumber width or the like is controlled based on the table.

### DESCRIPTION OF SYMBOLS

111, 211, 311: Sweeping signal generation unit
112, 212, 312: Laser driving unit
113, 213, 313, 413: Laser light source
114, 214, 314, 414: Photodetector
117, 217, 317: Pressure sensor
120, 220, 320: Control/analysis unit
130, 230, 330: Control unit
131, 231, 331: Laser control unit
140, 240, 340: Analysis unit
141, 241: Polynomial approximation unit
142, 242: Differential curve generation unit
143, 243, 345: Physical quantity determination unit
150, 250, 350: Storage unit
151, 251, 351: Table storage unit
110, 210, 310: Gas cell
344: Demodulation unit
361; Modulation signal generation unit
362: Adder
470: Engine
471: Cylinder
472: Piston
473: Combustion chamber
474a, 474b: Optical window
476; Crankshaft
477: Timing rotor
477a: Protrusion
478: Crank angle sensor

1. A gas absorption spectroscopic device comprising:
a laser light source with a variable wavenumber;
a photodetector configured to detect intensity of laser light emitted from the laser light source with a variable wavenumber and passed through a measurement target gas;
a laser driver configured to supply a driving current to the light source with a variable wavenumber to repeatedly sweep the wavelength of the laser light within a wavenumber range;
a pressure-related value acquisition unit configured to acquire a pressure-related value, the pressure-related value being a pressure value of the measurement target gas or a value varying in accordance with the pressure; and
a controller configured to control the laser driver to extend the wavenumber range for the sweep as the pressure increases based on the pressure-related value.

2. The gas absorption spectroscopic device as recited in claim 1, further comprising:
a table storage means in which a plurality of tables defining sweep waveforms each different in sweep wavenumber width is stored in association with the pressure-related value,
wherein the control means reads out a table corresponding to the pressure-related value acquired from the pressure-related value acquisition means among the plurality of tables from the table storage means and controls the laser driver in accordance with the table.

3. The gas absorption spectroscopic device as recited in claim 1,
further comprising:
a polynomial approximation unit configured to approximate a curve of a change of light intensity detected by the photodetector by an approximate polynomial within a predetermined wavenumber width at each point of a wavenumber;
a differential curve generation unit configured to generate an n^{th}-order differential curve of the curve including zero-order, based on a coefficient of each term of the approximate polynomial of each point; and
a physical quantity determination means configured to determine at least one of a temperature, a concentration, and partial pressure of a specific gas contained in the measurement target gas, based on the n^{th}-order differential curve including the zero-order,
wherein the polynomial approximation unit changes the wavenumber width to be approximated, depending on the pressure-related value acquired by the pressure-related value acquisition means.

4. The gas absorption spectroscopic device as recited in claim 1,
wherein the laser driver modulates the driving current at a predetermined modulation amplitude and a predetermined modulation frequency,
wherein the gas absorption spectroscopic device further comprises a demodulation means configured to extract a component of the modulation frequency or a harmonic component of the modulation frequency from a detection signal by the photodetector, and
wherein the control means further controls the laser driver to vary the modulation amplitude depending on the pressure-related value.

5. The gas absorption spectroscopic device as recited in claim 1,
wherein the measurement target gas is a gas in a combustion chamber of an engine, and
wherein the pressure-related value is a crank angle of the engine.
